(19) United States
(12) Patent Application Publication
(10) Pub. No.: US 2002/0187126 A1
(43) Pub. Date: Dec. 12, 2002

(54) Methods for viral oncoapoptosis in cancer therapy

(75) Inventors: John A. Blaho, New York, NY (US); Martine Aubert, New York, NY (US)

Correspondence Address: DARBY & DARBY P.C., P. O. BOX 5257, NEW YORK, NY, 10150-5257, US

(73) Assignee: Mount Sinai School of Medicine

(21) Appl. No.: 10/118,655

(22) Filed: Apr. 8, 2002

Related U.S. Application Data
(60) Provisional application No. 60/282,214, filed on Apr. 6, 2001.

(51) Int. Cl. C12N 15/869; A61K 48/00

(52) U.S. Cl. 424/93.2; 435/456

(57) ABSTRACT

The present invention provides an effective approach to inducing apoptosis of cancer cells, for anti-cancer therapy, using modified herpes viruses (HSV). The modification, deletion of an immediate early gene, results in a replication defective HSV (rdHSV). As a result of deletion of the immediate early gene, specifically ICP27 or ICP4, or both, the modified HSV is unable to complete its replication cycle while inducing apoptosis of the infected tumor cell. A particular advantage of this approach is that induction of apoptosis is specific for tumor cells, but not for normal cells. Moreover, the modified HSV can be engineered to contain a cancer therapeutic gene, i.e., it can act as a cancer therapeutic gene therapy vector, although it has potent anti-tumor activity on its own.

This application claims priority to U.S. provisional application Ser. No. 60/282,214, filed Apr. 6, 2001, which is incorporated herein by reference in its entirety.

### FIELD OF THE INVENTION

The present invention relates to the fields of molecular and cell biology generally, and more specifically, it addresses methods for inducing apoptosis in cancer cells. In particular, there are provided methods for effecting oncoapoptosis using a replication defective herpes simplex virus.

### BACKGROUND OF THE INVENTION

Normal tissue homeostasis is mediated through a balance of the rate of cell proliferation and normal cell death. Abnormal cell growth, a hallmark in the development of cancer as well as other cell proliferative disorders, is at least in part a result of a disruption or aberration in this balance. The effects of cancer are catastrophic, causing over half a million deaths per year in the United States alone.

Apoptosis, or programmed cell death, is a highly regulated process, that is activated during normal development and by various stimuli that disturb cell metabolism and physiology. Characteristic features of apoptosis include apoptotic body formation, cell shrinkage, membrane blebbing, chromatin condensation, and DNA fragmentation (Kerr et al., Br J Cancer. 1972, 26:239-57). These apoptotic features are the consequence of regulated proteolysis processes (Cryns and Yuan, Genes Dev. 1998, 12:1551-70; Salvesen and Dixit, Cell 1997, 91:443-6). The apoptotic potential of a cell has a profound effect on how that cell might respond to an environmental stress. In tumor cells, modulation of apoptosis plays a significant role in the progression to malignancy (Lowe and Lin, Carcinogenesis 2000, 21:485-95).

A central mediator of the cellular response to environmental stresses is the p53 protein, which is a major determinant of whether a cell will undergo apoptosis (Levine, Cell 1997, 88:323-31; Prives and Hall, J Pathol. 1999, 187:112-26). Mutations in the p53 gene are found in more than half of all human tumors (Hainaut et al., Nucleic Acids Res. 1998, 26:205-13; Hainaut and Hollstein, Adv Cancer Res. 2000, 77:81-137). Activation of p53 occurs in response to various types of stress including DNA damage, hypoxia, and oncogene activation, and results in increased levels of modified protein. Activated p53 initiates signaling pathways that lead to either cell cycle arrest or apoptosis (Levine, Cell 1997, 88:323-31; Prives and Hall, J Pathol. 1999, 187:112-26). The pro-apoptotic response of p53 to DNA damage introduced by chemotherapeutic agents or radiation is the basis for much of the efficacy of many of these treatments (Lowe et al, Cell 1993, 74:957-67).

Inactivation of p53 in cells is associated with resistance to anticancer drugs (Lowe et al., Science 1994, 266:807-10; Weinstein et al., Science 1997, 275:343-9). Given that over half of all human tumors comprise some defect or deficiency in wild-type p53, resistance to anti-cancer therapy resulting from such defects renders p53-deficient human tumors resistant to treatment. This problem is further compounded by the fact that any given tumor may contain a heterogeneous mixture of tumor cells, some of which are resistant to therapy and others which are not. Selective killing of only the tumor cells sensitive to the therapy leads to an overgrowth of tumor cells that are resistant to the chemotherapy.

Apoptosis also is an important mechanism of host cell defense against viral infections. Accordingly, several distinct viruses have developed mechanisms to block the premature apoptosis of infected cells (Koyama et al, Microbes Infect. 2000, 2:1111-7). These viral anti-apoptosis mechanisms delay cellular apoptosis that occurs as a result of viral infection, prolonging cell survival and increasing the yield of the viral progeny. Wild type (wt) herpes simplex virus 1 (HSV-1) triggers apoptosis in cells very early (less than one hour post infection) in infection and then subsequently (between 3 and 6 hours post infection) stimulates the synthesis of proteins that act to prevent the process from killing the infected cells (Aubert et al., J. Virol. 1999, 73:10359-70). HSV-1 replication is tightly regulated and occurs in an ordered cascade (Honess and Roizman, J. Virol. 1974, 14:8-19).

In addition to conventional strategies for the treatment of cancer (i.e., chemotherapy, radiotherapy, surgery, or combinations of these), which are known to have limited potential in the treatment of many cancers, certain human DNA viruses have been shown to kill tumor cells. For example, the E1b-mutant adenovirus ONYX-015 was recently shown in clinical trials to have anti-tumor activity, especially when administered in combination with chemotherapy (Ganly et al, Clin. Cancer Res. 2000, 6:798-806, Khuri et al., Nat. Med. 2000, 6:879-85). ONYX-015 replicates in and lyses cells that are p53-negative. Two herpes simplex viruses, G207 and 1716, have also recently been tested in clinical trials for anti-tumor activity (Markert et al., Gene Ther. 2000, 7:867-74). G207 is deleted for the viral neurovirulence ICP34.5 (Andreansky et al., Proc. Nat'l Acad. Sci. USA 1996, 93:11313-8; Chou et al, Science 1990, 250:1262-6) and ribonucleotide reductase genes while 1716 is only deleted for ICP34.5.

Despite the fact that the above DNA viruses have shown some potential as anti-cancer agents, all these prior virus based treatments rely on conditionally (or selectively) replicating viruses. Thus, productive viral replication is required to occur in the target cancer cells for each of these viruses to achieve tumor cell killing. The types of treatment regimens available to treat cancer using such viruses create significant dangers associated with administering such replication competent viral compositions to human subjects. These safety concerns make it preferable to administer viral compositions that are not replication competent, which will selectively kill tumor cells but are nevertheless unable to complete their replication cycle. To date, no such compositions are known.

Thus, many currently available chemotherapeutic and radiation cancer therapies are inadequate for effectively treating over half of human cancers and many alternative therapeutic options raise safety concerns that make them impractical. There clearly remains a need for more effective strategies of anti-cancer therapy for such cancers.

### SUMMARY OF THE INVENTION

The present invention advantageously provides an effective approach to inducing apoptosis of cancer cells, i.e., for anti-cancer therapy, using modified herpes viruses (HSV). The modification, deletion of an immediate early gene, results in a replication defective HSV (rdHSV), i.e., an HSV that cannot replicate in cells that it infects. Furthermore, as a result of deletion of the immediate early gene, specifically ICP27 or ICP4, or both, the modified HSV is unable to complete its replication cycle while inducing apoptosis of the infected tumor cell. A particular advantage of this approach is that induction of apoptosis is specific for tumor cells, but not for normal cells. Moreover, the modified HSV can be engineered to contain a cancer therapeutic gene, i.e., it can act as a cancer therapeutic gene therapy vector, although it has potent anti-tumor activity on its own.

Thus, in one embodiment, the invention provides a method of inducing apoptosis of a cancer cell. The method comprises contacting the cancer cell with a composition comprising a modified herpes simplex virus (HSV) as set forth above, e.g., having a defect in ICP27 (HSVΔ27) or having a defect in ICP4 (HSVΔICP4 ). Preferably the cancer cell in which the HSV composition induces apoptosis is resistant to anti-cancer therapy. For example, the cancer cell can be part of a solid tumor. The solid tumors cancer cell can be derived from a tissue, organ, or cell selected from the group consisting brain, lung, liver, spleen, kidney, lymph node, small intestine, blood cells, pancreas, colon, stomach, breast, endometrium, prostate, testicle, ovary, skin, head and neck, esophagus, bone marrow and blood. In various embodiments, the tumor is contacted with the HSVΔ27 or HSVΔICP4 before the anti-cancer therapy, after the anti-cancer therapy, or concurrently with the anti-cancer therapy.

Accordingly, the invention further provides a method of sensitizing a tumor to anti-cancer therapy. This method comprises contacting the tumor with a composition comprising HSVΔ27 or HSVΔICP4, wherein the tumor is more sensitive to treatment with an anti-cancer agent when contacted with the composition comprising the modified HSV than in the absence of the contacting with the modified HSV. The anti-cancer therapy can be a chemotherapeutic agent, a radioactive agent, a nucleic acid encoding a cancer therapeutic agent, or an anti-angiogenic agent.

In yet another embodiment, the invention provides a method of inducing apoptosis of a cancer cell comprising a defect in p53. The method comprises contacting the cancer cell with a HSVΔ27 or HSVΔICP4. Preferably such a cancer cell comprises a mutation in the p53 gene, a defect in the expression of p53 of the cells, a decrease in the post-translational phosphorylation of p53 yielding an under-phosphorylated p53 as compared to wild-type p53 in non-cancer cells, or an increase in the post-translational phosphorylation of p53 yielding an over-phosphorylated p53 as compared to wild-type p53 in non-cancer cells.

In still another embodiment, the modified HSVΔ27 further comprises a defect in an additional immediate early (IE) gene that triggers apoptosis but does not prevent apoptosis. In specific embodiments, the additional IE gene is ICP4 or gD, or both. In a more particular embodiment, the HSV 27 further comprises a defect in ICP4.

In still another embodiment, the invention provides a method of treating cancer in a mammal. The method comprises administering to the mammal a composition comprising HSVΔ27 or HSVΔICP4 in an amount effective to induce apoptosis of the cancer cells in the mammal. Preferably, the mammal is a human. In specific embodiments, the mammal has a cancer of a tissue, organ, or cell selected from the group consisting of brain, lung, liver, spleen, kidney, lymph node, small intestine, blood cells, pancreas, colon, stomach, breast, endometrium, prostate, testicle, ovary, skin, head and neck, esophagus, bone marrow and blood. In addition, the cancer can be in the form of an operable tumor, and the administering is performed before, during, or after the tumor is resected. As noted above, the operable tumor may be resistant to cancer therapy. Alternatively, the operable tumor may comprise cells that have a defective p53, e.g., as set forth above.

In the methods of treatment, HSVΔ27 or HSVΔICP4 can be administered to the mammal locally at the site of the tumor before, during or after resection. Alternatively, the modified HSV may be administered to the mammal systemically.

Still another embodiment of the treatment comprises administering an anti-cancer therapy with the modified HSV. The anti-cancer therapy can be a chemotherapeutic agent, a nucleic acid encoding a cancer therapeutic agent, radiation therapy, or an agent that affect the vasculature of a tumor. In accordance with the invention, the anti-cancer agent can be administered before, during, or after the administration of the HSVΔ27 or HSVΔICP4, e.g., locally at the site of the cancer. The cancer can be in the form of an operable tumor. The chemotherapeutic agent can be selected from the group consisting of cisplatin, 5-FU, mitomycin, etoposide, camptothecin, actinomycin-D, doxorubicin, verapamil, podophyllotoxin, daunorubicin, vincristine, vinblastine, melphalan cyclophosphamide, TNF-α, taxol and bleomycin. One clear advantage of this aspect of the invention involves treatment of tumors in which some cells are p53-positive while others are p53-negative. The HSVΔ27 and/or HSVΔICP4 therapeutic will target the p53 negative cells while the other approach, e.g., radiation or chemotherapy, targets the p53-positive cells. As a result, the radiation and/or chemotherapy will not simply select for resistant tumor cells.

The nucleic acid encoding a cancer therapeutic agent, which preferably is delivered by the modified HSV, may be an expression construct comprising a polynucleotide encoding a cancer therapeutic gene under the control of a promoter. Examples of cancer therapeutic gene include p21, p53, Rb, APC, DCC, NF-1, NF-2, BCRA2, p16, FHIT, WT-1, MEN-I, MEN-II, BRCA1, VHL, FCC and MCC. Examples of suitable promoters include SV40, CMV IE, RSV LTR, β-actin, HSV IE and HSV E. In another embodiment, instead of the modified HSV, the expression construct may be a retroviral vector, an adenoviral vector, an adenoassociated viral vector, a herpes simplex viral vector, an alphaviral vector, or a cytomegaloviral vector.

In the combination therapy with an agent that affects tumor vasculature, the the agent can be an anti-angiogenic agent selected from the group consisting of marimastat, COL-3, neovastat, thalidomide, squalamine, endostatin, angiostatin interferon-α, anti-VEGF antibody, or interleukin 12 (IL-12).

In the combination therapy with radiation therapy, the radiation therapy may be, but is not limited to, one or more of administering X-irradiation, UV-radiation, gamma-radiation, or microwave radiation. In a specific embodiment, the total dose of radiation is about 1 Gy to about 80 Gy. In another specific embodiment, the total does of x-ray radiation is between 200 and 6000 roentgens.

In the therapeutic methods of the invention, the modified HSV, i.e., HSVΔ27 or HSVΔICP4, along with any expression construct or chemotherapeutic agent, is administered to the tumor, e.g., through a catheter or a syringe, e.g., by direct injection into the tumor.

The accompanying Detailed Description, along with the Example and Drawings, further elaborate on and explain these and other aspects of the invention.

### DETAILED DESCRIPTION

Clinical resistance to anticancer agents is the principal reason for treatment failure in patients with cancer (Gottesman, Cancer Res. 1993, 53, 747-754). P53, a ubiquitous tumor suppressor, is known to be mutated or defective in over half of human cancers. It is now known that cancer cells in which the p53 has been inactivated are associated with resistance to anticancer drugs and radiation therapy. The present invention for the first time identifies a replication defective viral composition that is able to selective induce apoptosis in such cancer cells.

The inventors have demonstrated that recombinant HSV-1 strains, which initiate but can not complete their replication cycle due to a reduction in their expression of early and late genes, have the ability to induce apoptosis in cells that are of a mutant p53 phenotype. Such replication-defective (rd) viruses will therefore prove useful as anti-oncogenic agents based on their ability to trigger apoptosis in infected human cells while reducing the potential for viral infection.

As described in greater detail herein below, the present inventors have shown that human tumor cells are susceptible to death by rdHSV-1-induced apoptosis. The common feature among susceptible tumor cells is that they accumulated undermodified p53.

Thus, the modified state of p53 is a marker for susceptibility to apoptosis triggered by rdHSV-1. In addition, the levels of p53 in the sensitive cells were much less than that in an oncogene transformed cell. It is contemplated that cells in which p53 has been inactivated by a deletion or frameshift mutation also are susceptible to rdHSV-1-induced cell death. Consistent with the discovery that p53 deficiency correlates with susceptibility to rdHSV-1-induced apoptosis, primary cells that accumulate normal levels of modified p53 are unable to be triggered to undergo apoptosis by rdHSV-1. Thus, rdHSV-1 treatment will have a minimal adverse impact on normal cells in the treated subject.

The present invention describes methods for achieving virus-mediated oncoapoptosis using rdHSV. Such methods will be useful in promoting, augmenting or inducing apoptotic cell death of tumor cells that are resistant or refractory to traditional cancer therapy. The inventors have found that a rdHSV that has a defect in an immediate early (IE) gene such as infected cell protein 27 (ICP27) or infected cell protein 4 (ICP4) is able to trigger apoptosis in cells that have a mutant p53 phenotype. Methods and compositions for exploiting the therapeutic potential of these findings are discussed in further detail herein below.

### Herpes Simplex Virus

Herpes simplex viruses, designated with subtypes 1 and 2, are enveloped viruses that are among the most common infectious agents encountered by humans, infecting millions of human subjects worldwide. The present invention preferentially uses rdHSV-1 compositions to kill, inhibit or otherwise reduce the growth of tumor cells. In particular, the cells being treated by the viral compositions of the present invention are characterized by a deficiency in p53. To the extent that an understanding of HSV-1 may be necessary produce the compositions of the present invention, the following is a brief discussion of HSV-1 lytic replication.

HSV-1 is a neurotropic herpesvirus that causes a variety of infections in humans. HSV-1 has a broad host range and is able to infect and initiate replication in essentially all types of human cells. It remains latent in the neurons of its host for life and can be reactivated to cause lesions at or near the initial site of infection. Recurrent infections result from the lytic replication of the virus after reactivation from the latent state. During productive lytic infection in cultured cells, HSV-1 gene expression proceeds in a tightly regulated cascade (Roizman and Sears, In B. N. Fields and D. M. Knipe (ed.), Virology, 3rd ed. Lippincott-Raven, Philadelphia, Pa., 1996). Changes in the levels of HSV-1 gene expression during infection are usually the consequence of transcriptional regulation (Honess and Roizman, J. Virol. 1974, 14:8-19, Honess and Roizman, Proc. Nat'l Acad. Sci USA 1975, 72:1276-80). The first viral genes expressed during infection are transcribed in the absence of de novo viral protein synthesis and are termed the a or immediate-early (IE) genes. The α-gene products ICP0, 4, 22, and 27 have regulatory functions and cooperatively act to regulate the expression of all classes of viral genes. The β or early (E) genes are expressed next and encode many proteins involved in viral DNA synthesis such as the viral thymidine kinase (TK). The last genes expressed are the γ or late (L) genes and they mainly encode virion components such as VP16 and the virion host shutoff (vhs) protein.

HSV-1 is a member of a family of highly cytolytic viruses, the alphaherpesviridae, whose lytic replication cycle ultimately leads to the destruction of cells in culture. The cytopathic effect (CPE) of HSV-1 infection is generally observed as the rounding up of cells almost immediately upon infection and tends to become more severe with increasing times of infection. Manifestations of HSV-1 infection include the loss of matrix binding proteins on the cell surface leading to detachment; modifications of membranes; cytoskeletal destabilizations; nucleolar alterations, and chromatin margination/aggregation or damage, as well as a decrease in cellular macromolecular synthesis (Avitabile et al., J Virol. 1995, 69:7472-82; J Gen Virol. 1984, 65:1225-1228; Fenwick and McMenamin, J Gen Virol. 1978, 41:37-51; Heeg et al., Arch Virol. 1986, 91:257-70; Roizman, B. Proc Natl Acad Sci USA. 1962, 48:228-234; Roizman, and Roanne, Virology.1964, 22:262-269; Schek and Bachenheimer, J Virol. 1985, 55:601-610; Strom, and Frenkel, J Virol. 1987, 61:2198-2207). Thus, productive HSV-1 infection causes major biochemical alterations and has various structural ramifications.

The observed death of cells following wild type HSV-1 infection is likely due to some form of viral-induced necrosis, which leads to the classic manifestations of CPE. This cytopathology is a consequence of the virus “taking over the cell” in order to perform its replication cycle, as well as the presence of toxic viral gene products. The recognition that HSV-1 encodes gene products that might directly injure host cells has limited the development of the virus as a gene transfer vehicle. In fact, certain research efforts in this area have focused on limiting the synthesis of viral proteins in an attempt to reduce cell toxicity (Brehm et al., Virology 1999, 256:258-69; Johnson et al., J Virol. 1992, 66:2952-65; Johnson et al., J Virol. 1994, 68:6347-62; Samaniego et al, J Virol. 1998, 72:3307-20; Samaniego et al., J Virol. 1997, 71:4614-25). Supporting this contention are recent reports showing that HSV-1 recombinants that do not express any a regulatory proteins, appear to be devoid of toxicity (Brehm et al., Virology 1999, 256:258-69; Samaniego et al., J Virol. 1998, 72:3307-20; Samaniego et al., J Virol. 1997, 71:4614-25).

HSV-1 infection can induce programmed cell death in cultured cells through at least two separate pathways, which are distinct from the necrotic route described above. Initially, cell death caused by complete blockage of protein synthesis induced during infection was shown to be inhibited by the product of the γ₁34.5 gene (Chou et al., Science 1990, 250:1262-6; Chou and Roizman, Proc Natl Acad Sci U S A. 1992, 89:3266-70), which functions to block phosphorylation of the γ_{I}F-2 translation factor (He et al., J Biol Chem. 1998, 273:20737-43; He et al., Proc Natl Acad Sci U S A. 1997, 94:843-8). Recently, it has been shown that wild type HSV-1 infection could induce apoptosis under conditions in which de novo viral protein synthesis is inhibited, suggesting that (i) induction is likely an early event and (ii) HSV-1 produces polypeptides that specifically block apoptosis (Aubert and Blaho, J Virol. 1999, 73:2803-2813; Koyama and Adachi, J Gen Virol. 1997, 78:2909-12). HSV-1 infection induces apoptosis when cells are infected with viruses deleted for ICP27 or ICP4 or when infections occur in the presence of cycloheximide (Aubert and Blaho, supra; Aubert et al, J Virol. 1999, 73:10359-10370; Galvan et al., J Virol. 1999, 73:3219-3226; Galvan, and Roizman, Proc Natl Acad Sci U S A. 1998, 95:3931-6; Jerome et al., J Virol. 1999, 73:8950-7); Koyama and Adachi, J Gen Virol. 1997, 78:2909-12; Koyama and Miwa. J Virol. 1997, 71:2567-71). It is likely that HSV-1 induction of apoptosis occurs almost immediately upon infection and it is intriguing that the early viral protein kinase U_{S}3 and the late U_{S}5 and gD glycoproteins were reported to participate in blocking this effect (Jerome et al., J Virol. supra; Leopardi et al., Proc Natl Acad Sci U S A. 1997, 94:7891-6; Zhou et al, J Virol. 2000, 74:11782-11791).

In the present invention, it is demonstrated for the first time that HSV-1 infection induces apoptosis of tumor cells when such the tumor cells are infected with viruses deleted for ICP27 and/or ICP4. This induction of apoptosis is preferential for tumor cells that are characterized by a p53 deficiency, e.g., undermodification or mutation of p53. This finding is important because it provides for the first time an rdHSV-based treatment that is specific for tumor cells that are resistant to traditional anticancer agents.

The preferred viral compositions of the present invention that induce apoptosis comprise an ICP27-null virus. In other preferred embodiments, the virus may be an ICP4-null virus. Other viral compositions that may be useful in the present invention are those that have a deletion in both ICP27 and ICP4. Such replication defective viruses are known to those of skill in the art (Nguyen et al., J. Virol.1992, 66:7067-7072; Morrison and Knipe J. Virol.1994, 68:689-696; PCT Publication No. WO95/18852). Further, it should be understood that other viruses in which other immediate early genes that trigger apoptosis but do not prevent apoptosis also will be useful. U.S. Pat. No. 5,879,934, specifically incorporated herein by reference describes recombinant HSV vectors comprising genomic mutations within the ICP4 and ICP27 genes such that an ICP4 gene product and an ICP27 gene product are both defective. It is contemplated that such viruses will be useful in the methods of the present invention. U.S. Pat. No. 5,804,413 and U.S. Pat. No. 5,658,724 also disclose viruses defective in ICP4 and/or ICP27 that will be useful in the methods of the present invention.

### Monitoring p53 Status of Infected Cells

The results described in the Examples indicate that cells in which the p53 is undermodified are susceptible to apoptosis triggered by rdHSV-1 while those that accumulate phosphorylated p53 are resistant to such apoptosis. p53 is a phosphoprotein that predominantly localizes to the nucleus (Levine, Cell, 1997, 88:323-31, Prives and Hall J Pathol.1999, 187:112-26). In response to stress (e.g., genotoxic agent treatment), cellular signalling events lead to the phosphorylation of p53. It is known that phosphorylation of p53 at serine residue 15 occurs in response to genotoxic agents (Shieh et al., Embo J. 1999, 18:1815-23). p53 is a transcription factor that regulates the expression of numerous genes involved in the cellular stress response (Levine, Cell 1997, 88:323-31; Prives and Hall, J. Pathol. 1999, 187:112-26). Stress-induced phosphorylation of p53 at serine residue 392 (Lu et al., Proc Natl Acad Sci USA. 1998, 95: 6399-402) leads to the stimulation of DNA binding by p53, in turn, activating the protein (Hupp et al., Cell 1992, 71:875-86).

Greater than half of the missense point mutations that have been mapped in p53 from human tumor cells disrupt the protein's ability to bind DNA (Hainaut et al, Nucleic Acids Res. 1998, 26:205-13; Hamper and Elison, Nature 1994, 192:145-147). Thus, tumor cells that are defective for p53 may synthesize the full length protein, but this protein will no longer be functionally active. The instant invention indicated that loss of p53 function is required for susceptibility to apoptosis triggered by rdHSV-1 whereas cells that contain p53 which is functional for inducing growth arrest will be resistant to rdHSV-1 triggered apoptosis.

Thus, it is an aspect of the present invention that cells that have a mutant or modified p53 phenotype are susceptible to rdHSV-1 induced apoptosis. By mutant p53 phenotype, the present invention refers to a cell in which the p53 has a mutation that renders the p53 non-functional; alternatively the mutation is one which lowers the tumor suppressor activity of the p53 as compared to a wild-type p53. Mutant p53 phenotype also refers to those cells which have a reduced level of p53 expression as compared to normal cells of that lineage. Additionally, a mutant p53 phenotpe may be one in which the p53 is under-modified as compared to normal p53. In exemplary embodiments the p53 is under-phosphorylated as compared to wild-type p53.

In certain aspects of the invention, it may be necessary to determine the phosphorylation status of p53. Those of skill in the art know of various techniques for determining such a post-translational modification. For example, immunoblotting experiments using antibodies specific for p53 that is phosphorylated at either position 15 or 392 maybe performed. Anti-p53-Ser15 antibodies and anti-p53-Ser392 antibodies are commercially available from Oncogene Research (San Diego, Calif.). Cells that are unable to be triggered to undergo rdHSV-1 triggered apoptosis will likely produce p53 that is recognized by one or both of these p53-specific anti-phosphoserine antibodies.

Representative cellular markers for the p53 dependent growth arrest pathway can be analyzed using a variety techniques including Northern analysis with appropriate cDNA clones and Western blotting using commercially available antibodies. p21 which is able to bind and block a number of cyclin dependent kinase (cdk)-cyclin complexes leading to growth arrest can be followed for this purpose (Levine, Cell 1997, 88:323-31). In such studies, expression of p21 is seen in cells that are resistant to apoptosis triggered by rdHSV-1.

Measuring the kinase activities of cdk complexes in cell extracts also will be indicative cellular markers for the p53 dependent growth arrest pathway. Assays monitoring these activities use precipitates of cyclin A, cyclin E, and cdk2 and to test the precipitates' ability to phosphorylate an exogenous histone H1 substrate (Goodwin and DiMaio, Proc Natl Acad Sci USA. 1000, 97:12513-8). In such assays, cells which are not susceptible to rdHSV-1 triggered apoptosis have a functional p53 protein and exhibit a reduction in cdk activity as compared to those cells that have a non-functional p53 protein.

The consequence of p53 inducing p21, which then in turn inhibits cdk, is that p105Rb becomes hypophosphorylated. Ultimately, this form of p105Rb can complex with the E2F transcription factor leading to repression of E2F responsive genes which are required for cell cycle progression. Thus, determining the phosphorylation state p105Rb also reveals information about the p53 status of a cell relevant to the present invention in that cells resistant to rdHSV-1 triggered apoptosis have the ability to accumulate hypophosporylated p1-105Rb.

### Assays for Determining Apoptosis

Apoptosis, or programmed cell death, is a highly regulated process which is activated during normal development and by various stimuli that disturb cell metabolism and physiology (Kerr et al., Br J Cancer 1972, 26 239-57). Apoptotic signals converge to a central pathway involving a family of aspartate-specific cysteinyl proteases (cysteine aspartases or caspases), which are activated by proteolytic cleavage. This activation leads to the processing of various cytoplasmic and nuclear targets by a subclass of downstream effectors (which are also caspases) termed “executioners” (Green, Cell 1998, 94 695-698; Salvesen and Dixit, Cell 1997, 91 443-6; Villa et al., Trends Biochem Sci. 1997, 22 388-93).

Depending on the nature of the death signal, cells activate apoptotic events through two major pathways (Green, supra; Nagata, Cell 1997, 88 355-365; Sun et al., J Biol Chem. 1999, 274 5053-60). One is mediated via a death receptor, such as Fas or tumor necrosis factor (TNF) receptors. Once the receptor binds ligand, it then recruits an adaptor molecule that allows the binding and autocleavage/activation of procaspase-8. Activated caspase-8 induces a cascade, which includes processing of effector caspases (executioners) caspase-3 and caspase-7. In the other route, an apoptotic signal is transmitted to the mitochondria, resulting in release of cytochrome C into the cytoplasm, where it associates with Apaf-1 and permits the recruitment and activation of caspase-9 (Green, supra, Sun et al., supra). This, in turn, also leads to the cascade of events culminating in the activation of the executioners of apoptosis. In both pathways, caspase cleavage ultimately leads to the morphological and biochemical features characteristic of apoptosis, including apoptotic body formation, cell shrinkage, membrane blebbing, chromatin condensation, and DNA fragmentation (Kerr et al., supra). Among the cleavage targets are the DNA repair enzyme poly(ADP-ribose) polymerase (PARP) and the DNA fragmentation factor (DFF). Thus, the process of apoptosis generally involves the processing of caspase-3, DFF, and PARP (Salvesen and Dixit, supra; Villa et al., supra). Finally, it should be noted that both pathways can also engage in a self-amplifying process of mutual activation (Green, supra).

There are a number of HSV-1 proteins that are associated with apoptosis modulation. These include ICP27 (encoded by α27; Aubert and Blaho, J. Virol. 1999, 73:2803-13); ICP4 (encoded by α4; Leopardi and Roizman, Proc. Natl. Acad. Sci., USA 1996, 93:9583-7); ICP22 (encoded by α22; Aubert et al., J. Virol. 1999, 73:10359-70); Us3 (encoded by US3; Leopardi et al., Proc. Natl Acad. Sci. USA. 1997, 94:7891-6); vhs (encoded by UL41; Aubert et al., J. Virol. 1999, 73:10359-70); gJ (encoded by Us5, Jerome et al., J. Virol. 1999, 7273:8950-7); gD (encoded by Us6; Zhou et al., J. Virol. 2000, 74:11782-11791) and ICP34.5 (encoded by γ₁34.5; Chou and Roizman, Proc. Natl. Acad. Sci. USA 1992, 89: 3266-70). Of these, the proteins required for apoptosis prevention are: ICP27; ICP4 and gD; ICP22 and Us3 and gJ are accessory proteins in apoptosis prevention. As indicated elsewhere herein, it is contemplated that useful viruses for the present invention will be those that are deleted in proteins that are required for apoptosis prevention. A particularly preferred virus is one that has been deleted in ICP27; another preferred virus is one that has been deleted in ICP4; however, other viruses which are deleted in one or more of the proteins listed above will also be particularly useful for inducing apoptosis in cancer cells in the present invention.

The defining features of the apoptosis process are chromatin condensation (pyknosis), fragmentation of nuclei (karyorhexis), specific nucleosomal laddering, membrane blebbing, and the formation of apoptotic bodies. Assays for monitoring apoptosis are well known to those of skill in the art and include for example, monitoring cell shrinkage, nuclear condensation, monitoring appearance of genomic DNA fragmentation ladders; monitoring the processing of PARP, a 116 kDa protein, which generates an 85 kDa product which may be detected by the anti-PARP antibody (Aubert et al., J. Virol. 1999, 73:10359-70); and monitoring apoptosis-induced processing of DFF (45 kDa) and caspase-3 (32 kDa) as determined by the loss of reactivity with the anti-DFF and anti-caspase-3 antibodies.

### Methods of Treating Cancer

The present invention involves the treatment of cancers, characterized by resistance to traditional therapies, using rdHSV-1 compositions of the present invention. In particular, it is contemplated that the cancers treatable by the present invention are those that have a defect in p53 as described herein above. Thus, it is contemplated that a wide variety of tumors may be treated using the present invention, including cancers of the brain (glioblastoma, astrocytoma, oligodendroglioma, ependymomas), lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that all the tumor cells be killed or induced to undergo apoptosis. Rather, to accomplish a meaningful treatment, all that is required is that the tumor growth be slowed to some degree or localized to a specific area and inhibited from spread to disparate sites. It may be that the tumor growth is completely blocked, however, or that some tumor regression is achieved. Clinical terminology such as “remission” and “reduction of tumor” burden also are contemplated as these terms are conventionally understood in the art.

The viral compositions described in the present invention will be used for the therapeutic intervention of the disorders discussed herein above as well as any other neoplastic disorders that are refractory to traditional therapies. The viral compositions may be formulated into appropriate formulations for administering to a subject. Formulations would be selected based on the route of administration and purpose including, but not limited to, liposomal formulations and classic pharmaceutical preparations.

Administration of the compositions can be systemic or local and may comprise a single site injection of a therapeutically effective amount of the rdHSV-1 composition of the present invention. Any route known to those of skill in the art for the administration of a therapeutic composition of the invention is contemplated, including for example, intravenous, intramuscular, subcutaneous, or a catheter for long term administration. Alternatively, it is contemplated that the viral therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a periodic basis, for example, daily, weekly or monthly.

The viruses of the present invention are contacted with the cancer cell. In additional embodiments, it is contemplated that the therapeutic composition also may contain an expression construct comprising an additional therapeutic gene. For these embodiments, an exemplary expression construct comprises a virus or engineered construct derived from a viral genome. The expression construct generally comprises a nucleic acid encoding the gene to be expressed and also additional regulatory regions that will effect the expression of the gene in the cell to which it is administered. Such regulatory regions include for example promoters, enhancers, polyadenylation signals and the like.

It is now widely recognized that DNA may be introduced into a cell using a variety of viral vectors. In such embodiments, expression constructs comprising viral vectors containing the genes of interest may be adenoviral (see for example, U.S. Pat. No. 5,824,544; U.S. Pat. No. 5,707,618; U.S. Pat. No. 5,693,509; U.S. Pat. No. 5,670,488; U.S. Pat. No. 5,585,362; each incorporated herein by reference), retroviral (see for example, U.S. Pat. No. 5,888,502; U.S. Pat. No. 5,830,725; U.S. Pat. No. 5,770,414; U.S. Pat. No. 5,686,278; U.S. Pat. No. 4,861,719 each incorporated herein by reference), adeno-associated viral (see for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479 each incorporated herein by reference), an adenoviral-adenoassociated viral hybrid (see for example, U.S. Pat. No. 5,856,152 incorporated herein by reference) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688 each incorporated herein by reference) vector. Preferred vectors are modified (HSV Δ27 and/or HSVΔICP4) viruses of the invention.

In other embodiments, non-viral delivery of the additional therapeutic gene is contemplated. These include calcium phosphate precipitation (Graham and Van Der Eb, Virology 1973, 52:456-467; Chen and Okayama, Mol. Cell Biol. 1987, 7:2745-2752; Rippe et al., Mol. Cell Biol. 1990, 10:689-695), DEAE-dextran (Gopal, Mol. Cell Biol. 1985, 5:1188-1190), electroporation (Tur-Kaspa et al., Mol. Cell Biol. 1986, 6:716-718; Potter et al., Proc. Nat. Acad. Sci. USA, 1984, 81:7161-7165), direct microinjection (Harland and Weintraub, J. Cell Biol., 1985, 101:1094-1099), DNA-loaded liposomes (Nicolau and Sene, Biochim. Biophys. Acta 1982, 721:185-190; Fraley et al., Proc. Natl. Acad. Sci. USA 1979, 76:3348-3352; Felgner, Sci Am. 1997, 276(6):102-6; Felgner, Hum Gene Ther. 1996, 7(15):1791-3), cell sonication (Fechheimer et al., Proc. Natl. Acad. Sci. USA 1987, 84:8463-8467), gene bombardment using high velocity microprojectiles (Yang et al., Proc. Natl. Acad. Sci USA 1990, 9568-9572), receptor-mediated transfection (Wu and Wu, J. Biol. Chem. 1987, 262:4429-4432; Wu and Wu, Biochemistry 1988, 27:887-892; Wu and Wu, Adv. Drug Delivery Rev. 1993, 12:159-167) and electrotransfer (PCT Publication Nos. WO99/01157, WO99/01158, WO99/01175).

In a particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver diseases, targeted diagnosis and therapy using specific receptors and ligands, Wu G, Wu C ed., New York: Marcel Dekker, pp. 87-104, 1991). The addition of DNA to cationic liposomes causes a topological transition from liposomes to optically birefringent liquid-crystalline condensed globules (Radler et al., Science 1997, 275(5301):810-4). These DNA-lipid complexes are potential non-viral vectors for use in gene therapy and delivery.

Liposome-mediated nucleic acid delivery and expression of foreign DNA in vitro has been very successful. Also contemplated in the present invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda et al., Science 989, 243:375-378). In other embodiments, the liposome may be complexed or employed in conjunction with nuclear nonhistone chromosomal proteins (HMG-1) (Kato et al, J. Biol. Chem. 1991, 266:3361-3364). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems that can be employed to deliver a nucleic acid encoding a therapeutic gene into cells include receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993, supra).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987, supra) and transferrin (Wagner et al, Proc. Nat'l. Acad Sci. USA 1990, 87(9):3410-3414). Recently, a synthetic neoglycoprotein, which recognizes the same receptor as ASOR, has been used as a gene delivery vehicle (Ferkol et al, FASEB J.1993, 7:1081-1091; Perales et al., Proc. Natl. Acad. Sci., USA 1994, 91:4086-4090) and epidermal growth factor (EGF) has also been used to deliver genes to squamous carcinoma cells (Myers, EPO 0273085).

In other embodiments, the delivery vehicle may comprise a ligand and a liposome. For example, Nicolau et al (Methods Enzymol. 1987, 149:157-176) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a particular cell type by any number of receptor-ligand systems with or without liposomes.

In another embodiment of the invention, the expression construct may simply consist of naked recombinant DNA or plasmids. Transfer of the construct may be performed by any of the methods mentioned above that physically or chemically permeabilize the cell membrane. This is applicable particularly for transfer in vitro, however, it may be applied for in vivo use as well. Dubensky et al. (Proc. Nat. Acad. Sci. USA 1984, 81:7529-7533) successfully injected polyomavirus DNA in the form of CaPO₄ precipitates into liver and spleen of adult and newborn mice demonstrating active viral replication and acute infection. Benvenisty and Neshif (Proc. Nat. Acad. Sci. USA 1986, 83:9551-9555) also demonstrated that direct intraperitoneal injection of CaPO₄ precipitated plasmids results in expression of the transfected genes.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Nature 1987, 327:70-73). Several devices for accelerating small particles have been developed. One such device relies on a high voltage discharge to generate an electrical current, which in turn provides the motive force (Yang et al., Proc. Natl. Acad. Sci USA 1990, 87:9568-9572). The microprojectiles used have consisted of biologically inert substances such as tungsten or gold beads.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various tumor types. The section below on routes contains an extensive list of possible routes. For practically any tumor, systemic delivery is contemplated. This will prove especially important for attacking microscopic or metastatic cancer. Where discrete tumor mass may be identified, a variety of direct, local and regional approaches may be taken. For example, the tumor may be directly injected with the expression vector or protein. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

### Combined Therapy with Traditional Chemotherapy or Radiotherapy

Tumor cell resistance to DNA damaging agents represents a major problem in clinical oncology. One goal of current cancer research is to find ways to improve the efficacy of chemo- and radiotherapy. One way is by combining such traditional therapies with gene therapy. For example, the herpes simplex-thymidine kinase (HS-tk) gene, when delivered to brain tumors by a retroviral vector system, successfully induced susceptibility to the antiviral agent ganciclovir. One embodiment of the present invention includes administration of the rdHSV compositions in conjunction with chemo- or radiotherapeutic intervention, immunotherapy, or with other anti-angiogenic/anti-lymphangiogenic therapy.

To kill cells, inhibit cell growth, inhibit metastasis, inhibit angiogenesis or otherwise reverse or reduce the malignant phenotype of tumor cells, using the methods and compositions of the present invention, one would generally contact a “target” cell, a tumor or its vasculature, with the rdHSV viral therapeutic compositions of the present invention and at least one other agent. These compositions would be provided in a combined amount effective to kill or inhibit proliferation of the cancer by killing and/or inhibiting the proliferation of the cancer cells. This process may involve contacting the cells with the viral composition and the agent(s) or factor(s) at the same time. This may be achieved by contacting the cell with a single composition or pharmacological formulation that includes both agents, or by contacting the cell with two distinct compositions or formulations, at the same time, wherein one composition includes the virus of the present invention and the other includes the second agent.

Alternatively, the therapeutic treatment employing the viral compositions described herien may precede or follow the other agent treatment by intervals ranging from minutes to weeks. In embodiments where the other agent and viral composition are administered separately, one would generally ensure that a significant period of time did not expire between the time of each delivery, such that the agent and the viral-based therapeutic would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. Repeated treatments with one or both agents is specifically contemplated. In specific embodiments, an anti-cancer therapy may be delivered that directly attacks the cancer cells in a manner to kill, inhibit or necrotize the cancer cell. In addition, a therapeutic composition based on the viral compositions of the present invention also is administered to the individual in amount effective to have an apoptotic effect. The virus compositions may be administered following the other anti-cancer agent, before the other anti-cancer agent or indeed at the same time as the other anti-cancer agent.

Agents or factors suitable for use in a combined therapy are any chemical compound or treatment method that induces DNA damage when applied to a cell. In treating cancer according to the invention, one would contact the tumor cells and/or the endothelia of the tumor vessels with an agent in addition to the therapeutic virus bases compositions of the present invention. Such agents and factors include radiation and electromagnetic energy waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. Kinase inhibitors also contemplated to be useful in combination therapies with the virus based therapeutic compositions of the present invention. The agent may be prepared and used as a combined therapeutic composition, or kit, by combining it with a HSV-1 virus deleted for ICP27 and/or ICP4 as described by the present invention. The invention also encompasses the use of a combination of one or more DNA damaging agents, whether radiation-based or actual compounds, such as the use of X-rays with cisplatin or the use of cisplatin with etoposide.

Agents that directly cross-link nucleic acids, specifically DNA, are envisaged to facilitate DNA damage leading to a synergistic, antineoplastic combination with HSV-1 virus-based therapy. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

By way of example the following is a list of chemotherapeutic agents and the cancers which have been shown to be managed by administration of such agents. Combinations of these chemotherapeutics with the virus-based compositions of the present invention may prove to be useful in amelioration of various neoplastic disorders. Examples of these compounds include adriamycin (also known as doxorubicin), VP-16 (also known as etoposide), and the like, daunorubicin (intercalates into DNA, blocks DNA-directed RNA polymerase and inhibits DNA synthesis); mitomycin (also known as mutamycin and/or mitomycin-C) is an antibiotic isolated from the broth of Streptomyces caespitosus which has been shown to have antitumor activity; Actinomycin D also may be a useful drug to employ in combination with the viruses of the present invention because tumors which fail to respond to systemic treatment sometimes respond to local perfusion with dactinomycin which also is known to potentiate radiotherapy. It also is used in combination with primary surgery, radiotherapy, and other drugs, particularly vincristine and cyclophosphamide and has been found to be effective against Ewing's tumor, Kaposi's sarcoma, and soft-tissue sarcomas, choriocarcinoma, metastatic testicular carcinomas, Hodgkin's disease and non-Hodgkin's lymphomas.

Bleomycin is a mixture of cytotoxic glycopeptide antibiotics isolated from a strain of Streptomyces verticillus, is effective in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Cisplatin has been widely used to treat cancers such as metastatic testicular or ovarian carcinoma, advanced bladder cancer, head or neck cancer, cervical cancer, lung cancer or other tumors and may be a useful combination with the virus-based therapeutic compositions of the present invention. VP16 (etoposide) and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS). Tumor Necrosis Factor [TNF; Cachectin] is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. TNF can be quite toxic when used alone in effective doses, so that the optimal regimens probably will use it in lower doses in combination with other drugs. Its immunosuppressive actions are potentiated by γ-interferon, so that the combination potentially is dangerous. A hybrid of TNF and interferon-α also has been found to possess anti-cancer activity.

Taxol an antimitotic agent original isolated from the bark of the ash tree, Taxus brevifolia, and its derivative paclitaxol have proven useful against breast cancer; these compounds and other anti-tumor taxoids may be used in the combination therapies of the present invention. Beneficial responses to vincristine have been reported in patients with a variety of other neoplasms, particularly Wilms' tumor, neuroblastoma, brain tumors, rhabdomyosarcoma, and carcinomas of the breast, bladder, and the male and female reproductive systems. Vinblastine also is indicated as a useful therapeutic in the same cancers as vincristine. The most frequent clinical use of vinblastine is with bleomycin and cisplatin in the curative therapy of metastatic testicular tumors. It is also active in Kaposi's sarcoma, neuroblastoma, and Letterer-Siwe disease (histiocytosis X), as well as in carcinoma of the breast and choriocarcinoma in women.

Melphalan also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. Melphalan is the active L-isomer of the D-isomer, known as medphalan, which is less active against certain animal tumors, and the dose needed to produce effects on chromosomes is larger than that required with the L-isomer. Melphalan is available in form suitable for oral administration and has been used to treat multiple myeloma. Available evidence suggests that about one third to one half of the patients with multiple myeloma show a favorable response to oral administration of the drug. Melphalan has been used in the treatment of epithelial ovarian carcinoma.

Cyclophosphamide is stable in the gastrointestinal tract, tolerated well and effective by the oral and parental routes and does not cause local vesication, necrosis, phlebitis or even pain. Chlorambucil, a bifunctional alkylating agent of the nitrogen mustard type, has been found active against selected human neoplastic diseases. Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the regional delivery of virus-based therapeutic to patients with therapy resistant cancers will be a very efficient method for counteracting the clinical disease. Similarly, the chemo- or radiotherapy may be directed to a particular, affected region of the subjects body. Alternatively, systemic delivery of the virus and/or the agent may be appropriate in certain circumstances, for example, where extensive metastasis has occurred.

In addition to combining the virus-based therapies with chemo- and radiotherapies, it also is contemplated that combination with gene therapies will be advantageous. For example, targeting of virus-based therapies and p53 or p16 mutations at the same time may produce an improved anti-cancer treatment. Any other tumor-related gene conceivably can be targeted in this manner, for example, p21, Rb, APC, DCC, NF-1, NF-2, BCRA2, p16, FHIT, WT-1, MEN-I, MEN-II, BRCA1, VHL, FCC, MCC, ras, myc, neu, raf erb, src, fms, jun, trk, ret, gsp, hst, bcl and abl.

In addition to the anticancer therapeutics discussed above, it is contemplated that the viral compositions of the invention may be combined with angiogenesis inhibitors. An Internet site, cancertrials.nci.nih.gov/news/angio, is maintained by the National Institutes of Health which provides current information on the trials presently being conducted with anti-angiogenic agents. These agents include, for example, Marimastat (British Biotech, Annapolis Md.; indicated for non-small cell lung, small cell lung and breast cancers); AG3340 (Agouron, LaJolla, Calif.; for glioblastoma multiforme); COL-3 (Collagenex, Newtown Pa.; for brain tumors); Neovastat (Aeterna, Quebec, Canada; for kidney and non-small cell lung cancer); BMS-275291 (Bristol-Myers Squibb, Wallingford Conn.; for metastatic non-small cell ling cancer); Thalidomide (Celgene; for melanoma, head and neck cancer, ovarian, metastatic prostate, and Kaposi's sarcoma; recurrent or metastatic colorectal cancer (with adjuvants); gynecologic sarcomas, liver cancer; multiple myeloma; CLL, recurrent or progressive brain cancer, multiple myeloma, non-small cell lung, nonmetastatic prostate, refractory multiple myeloma, and renal cancer); Squalamine (Magainin Pharmaceuticals Plymouth Meeting, Pa.; non-small cell cancer and ovarian cancer); Endostatin (EntreMed, Rockville, Md.; for solid tumors); SU5416 (Sugen, San Francisco, Calif.; recurrent head and neck, advanced solid tumors, stage IIIB or IV breast cancer; recurrent or progressive brain (pediatric); Ovarian, AML; glioma, advanced malignancies, advanced colorectal, von-Hippel Lindau disease, advanced soft tissue; prostate cancer, colorectal cancer, metastatic melanoma, multiple myeloma, malignant mesothelioma: metastatic renal, advanced or recurrent head and neck, metastatic colorectal cancer); SU6668 (Sugen, San Francisco, Calif.; advanced tumors); interferon-α; anti-VEGF antibody (National Cancer Institute, Bethesda Md.; Genentech, San Franscisco, Calif.; refractory solid tumors; metastatic renal cell cancer, in untreated advanced colorectal); EMD121974 (Merck KCgaA, Darmstadt, Germany; HIV related Kaposi's Sarcoma, progressive or recurrent Anaplastic Glioma ); Interleukin-12 (Genetics Institute, Cambridge, Ma.; Kaposi's sarcoma); and IM862 (Cytran, Kirkland, Wash.; ovarian cancer, untreated metastatic cancers of colon and rectal origin and Kaposi's sarcoma). The parenthetical information following the agents indicates the cancers against which the agents are being used in these trials. It is contemplated that any of these disorders may be treated with the viruses of the present invention either alone or in combination with the agents listed as long as these disorders exhibit a defect in p53.

### Formulations and Routes for Administration to Patients

The therapeutic viral compositions for clinical use are preferably prepared as pharmaceutical compositions, i.e., in a form appropriate for in vivo applications. Generally, this entails preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the virus vector, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention include classic pharmaceutical preparations. Administration of these compositions according to the present invention will be via any common route so long as the target tissue is available via that route. The pharmaceutical compositions may be introduced into the subject by any conventional method, e.g., by intravenous, intradermal, intramuscular, intramammary, intraperitoneal, intrathecal, retrobulbar, intrapulmonary (e.g., term release); by sublingual, nasal, anal, vaginal, or transdermal delivery, or by surgical implantation at a particular site. A preferred route of administration for cancer therapy is intratumoral. The treatment may consist of a single dose or a plurality of doses over a period of time.

Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum-drying and freeze-drying techniques that yield a powder of the active ingredient plus any additional desired ingredient from a previously sterile-filtered solution thereof.

For oral administration the compositions of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. Parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood clotting levels in conjunction with relevant dose-response data. The final dosage regimen will be determined by the attending physician, considering factors that modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

In gene therapy embodiments employing viral delivery, the unit dose may be calculated in terms of the dose of viral particles being administered. Viral doses include a particular number of virus particles or plaque forming units (pfu). For embodiments involving virus, particular unit doses include 10³, 10⁴, 10⁵, 10⁶, 10⁷, 10⁸, 10⁹, 10¹⁰, 10¹¹, 10¹², 10¹³ or 10¹⁴ pfu. Particle doses may be somewhat higher (10 to 100-fold) due to the presence of infection defective particles.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals such as cows, sheep, pigs, horses and goats; companion animals such as dogs and cats; exotic and/or zoo animals; laboratory animals including mice rats, rabbits, guinea pigs and hamsters; and poultry such as chickens, turkeys, ducks and geese.

### EXAMPLES

The present invention is described in more detail with reference to the following non-limiting examples which represent preferred embodiments of the invention. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods which are disclosed and still obtain a like or similar result without departing from the spirit and scope of the invention.

### Example 1

### Modified HSV Induces Apoptosis of Human Tumor Cells

This Example describes exemplary materials and methods for all the Examples and initial results.

### Materials and Methods

Cells and viruses. All cells were maintained in Dulbecco's modified Eagle's medium (DMEM) containing 5% fetal bovine serum. Human larynx epidermoid carcinoma HEp-2 (Moore, Cancer Res.1955, 15:598-605), epithelial cervical carcinoma HeLa S3 (Puck, et al, J. Exp. Med.1956, 103:273-284, osteosarcoma 143B (Post and Roizman, Cell 1981, 25:227-32), and African green monkey kidney Vero (Yasumura and Kawakita, Nippon Rinsho 1963, 21:1209) cells were obtained from the American Type Culture Collection (Rockville, Md. V2.2 is a derivative Vero cell line, which carries a stably integrated copy of the ICP27 gene (Rice and Knipe, J Virol. 1990, 64:1704-15). Adenovirus type 5 transformed human primary embryonal kidney 293 (Graham et al., J Gen Virol. 1977, 36:59-74) and SV40 T antigen expressing 293T cells were obtained from the ATCC. Transformed 293 cells contain an integrated portion of the Adenovirus 5 early region 1 that facilities the synthesis of viral E1a and E1b (55 kDa and 19 kDa) proteins in these cells (Spector, Virology. 1983, 130:533-8). Primary C57B mouse embryo fibroblast cells also were used. Cell monolayers were infected at a multiplicity of infection (MOI) of 10 PFU/cell and the infections were maintained at 37° C. in DMEM with 5% newborn calf serum (NBCS) for 24 hours.

KOS1.1 virus (Hughes and Munyon, J Virol. 1975, 16:275-83) is the wild type HSV-1 (KOS) used in this study and vBSΔ27 virus is a recombinant HSV-1 (Δ27) in which the ICP27 gene was replaced by the β-galactosidase gene (Rice and Knipe, J Virol. 1990, 64:1704-15), i.e., HSVΔ27. CgalΔ3 virus (Johnson et al., J Virol. 1992, 66:2952-65) is a recombinant HSV-1 (Δ3) carrying a 4.6 kb insertion of a HCMVpromoter-lacZ gene within the ICP4 coding region, i.e., HSVΔICP4.

UV irradiation of cells. The technique for UV-treatment of infected cells is a modification of a previously described protocol (Aubert et al., J. Virol. 1999, 73:10359-70). Confluent HEp-2 cells grown in a 35 mm dish (Falcon) in 5% NBCS were placed on ice at a distance of 10 cm from a germicidal lamp (Model MR-4-60 Hertz, George W. Gates and Company, Franklin Square, N.Y.). Cells were exposed to UV light for 5 min. Following UV-treatment, cells were returned to 37° C. and incubated for varying times up to maximum of 6 hours, prior to preparing whole cell extracts.

Biochemical induction of apoptosis. Cell apoptosis was induced by the addition of staurosporine (Calbiochem, San Diego, Calif.) to the medium at a final concentration of either 1 or 2 μM. Cells were maintained in the presence of staurosporine for 24 h. TNF (Sigma) was added directly to medium at a final concentration of 10 ng/ml in combination with cycloheximide (Sigma) at a final concentration of 10 μg/ml for 24 h.

Extraction of cells and denaturing gel electrophoresis. Whole extracts of cells were obtained as previously described (Aubert and Blaho, J Virol. 1999, 73:2803-2813; Aubert et al., J. Virol. 1999, 73:10359-70) Protein concentrations were measured using a modified Bradford assay (Biorad) as recommended by the vendor. Equal amounts of infected cell proteins (50 μg) were separated in denaturing 15% DATD-acrylamide gels and electrically transferred to nitrocellulose membranes in a tank apparatus (Biorad) prior to immunoblotting with various primary antibodies. Unless otherwise noted in the text, all biochemical reagents were obtained from Sigma. Nitrocellulose was obtained directly from Schleicher and Schuell. Prestained protein molecular weight markers were purchased from Life Technologies.

Immunological reagents. Immunoblotting experiments were performed to detect cellular apoptotic proteins using mouse anti-caspase-3 monoclonal antibody (1:1,000; Transduction Laboratories Inc.) and mouse anti-PARP monoclonal antibody (1:2,000; Pharmingen) as described previously (Aubert et al, supra). Secondary (goat) anti-rabbit, anti-mouse and (rabbit) anti-goat antibodies conjugated with alkaline phosphatase (1:1,000) were purchased from Southern Biotech (Birmingham, Ala.) and secondary (goat) anti-mouse conjugated to horseradish peroxidase (1:1,000) was from Amersham. Anti-p53 (14211A) monoclonal antibody was obtained from Pharmingen. Anti-p53 (Ab-2, Calbiochem) and anti-pRb (G3245, Pharmingen) monoclonal antibodies were obtained from their suppliers.

DNA laddering by ethidium bromide staining. Low molecular weight DNA was isolated from cells as described (Koyama and Miwa, J. Virol. 1997, 71:2567; Aubert and Blaho, J. Virol. 1999, 73:2803). The DNA samples were subjected to electropheresis in a horizontal 1.5% agarose gel, followed by staining with 10 μg/ml ethidium bromide. DNA was visualilzed by UV light transillumination and photographed with Polaroid 67 film.

Microscopic analyses and computer graphics. Infected cell phenotypes were documented by phase-contrast light microscopy using an Olympus CK2/PM-10AK3 system with an attached 35 mm camera. For analyses of chromatin condensation, cells were grown and infected (as described above) in a 35-mm plate. At 24 hours post infection, the cells were incubated with the DNA dye Hoechst 33258 (Sigma) at a final concentration of 5 μg/ml in PBS for 15 minutes at 37° C. Immunoblots, autoradiograms, and 35 mm slides were digitized at 600 to 1200 dots per inch (d.p.i.) resolution using an AGFA Arcus II scanner linked to a Macintosh G3 PowerPC workstation. Raw digital images were saved as tagged image files (TIF) using Adobe Photoshop version 5.0.

### Results

Human cervical carcinoma HeLa cells present apoptotic features including cell shrinkage, membrane blebbing, and the formation of apoptotic bodies following infection with a recombinant HSV-1 (HSVΔ27) lacking the essential ICP27 regulatory protein (Aubert and Blaho, supra). HeLa cells contain integrated human papilloma virus (HPV) 18 DNA (Boshart et al., EMBO J. 1984, 3:1151-7) and express the viral E6 and E7 gene products (Schwarz et al., Nature 1985, 314:111-4). While most cell lines derived from cervical carcinomas possess intact genomic copies of p53 and p105_{Rb} genes (Scheffner, et al., Proc Natl Acad Sci USA 1991, 88:5523-7), these proteins are inactivated by E6 and E7, respectively (Munger et al., EMBO J. 1989, 8:4099-105; Scheffner et al., Cell 1990, 63:1129-36). The E6 protein acts to increase the ubiquitin-dependent degradation of p53 and E7 complexing to p105^{Rb} results in the release of the transcription factor, E2F, which in turn activates genes involved in cell proliferation (Blaho and Aaronson, Proc Natl Acad. Sci USA 1999, 96:7619-21; Howley, Cancer Res. 1991, 51:5019s-5022s; Laimins, Infect. Agents Dis. 1993, 2:74-86; Levine, Cell 1997, 88:323-31; Prives and Hall, J. Pathol.1999, 187:112-126; Villa, Adv. Cancer Res.1997, 71:321-41). Thus, the consequence of E6 and E7 expression in HeLa cells is the loss of check point control leading to immortalization. The goal was to determine whether human tumor cells are specifically sensitive to herpes simplex virus induced apoptosis (oncoapoptosis) and if p53 inactivation is a determinant of susceptibility.

Three human tumor cell lines were treated with replication-defective HSV-1 (rdHSV-1) strains for 24 h and apoptosis was defined by the appearance of death factor processing (Aubert et al., supra). Human 143B cells are an osteogenic murine sarcoma virustransformed line (Rhim et al., Int. J, Cancer 1975, 15:23-9, 1975) that was selected in bromodeoxyuridine for the loss of thymidine kinase (Bacchetti and Graham, Proc. Natl Acad Sci. USA 1977, 74:1590-4). Human epidermoid HEp-2 cells were derived from a patient with laryngeal carcinoma (Moore, Cancer Res. 1955, 15:598-605). Infection of HeLa, 143B, and HEp-2 cells with rdHSV-1 (HSVΔ27) leads to apoptosis, since PARP and procaspase-3 processing was detected as evidenced by the appearance of low molecular-weight bands on an immmunoblot, while wild-type (wt) virus (KOS1.1) and mock infection does not. In addition, infection of Hep-2 cells with a construct containing an insertion in the IPC4 coding region (Cga1Δ3, i.e., HSVΔICP4), which is also replication defective, also resulted in apoptosis. These results indicate that at least two separate rdHSV-1 strains can trigger apoptotic death in transformed human cells. Based on the current model for defining apoptosis during HSV-1 infection (Aubert et al., J. Virol. 2001, 75:1013-1030), it is concluded that these rdHSV-1 viruses are unable to prevent the induced cell death process from killing the infected cells, leading to the state of viral oncoapoptosis

HEp-2 cells are phenotypically similar to HeLa cells (Moore, supra) but have not been as extensively studied. While HPV30 has been isolated from human laryngeal carcinoma tissue specimens (Kahn et al., In. J. Cancer 1986, 37:61-5), there is currently no evidence that HEp-2 cells contain integrated papilloma virus DNA. To assess whether HEp-2 cells contain functional p53, cells were UV irradiated (Lu et al., Proc Natl Acad. Sci USA 1998, 95:6399-6402) and immune reactivities of p53 and death factors PARP and caspase-3 were measured. Activation of caspase-3 was detected as early a 30 min following UV treatment, as evidenced by the presence of a 29 kD band on an immunoblot, and PARP processing was observed beginning at 4 h post treatment, as evidenced by the presence of both the 116 and 85 kD bands. Detection of the processing of both of these death factors continued to increase up to the latest time point tested, 6 hours post treatment. In HEp-2 cells analyzed immediately after treatment, two forms of p53 were detected as evidenced by a doublet band, with the faster migrating form being the more abundant form. The detection of multiple forms of p53 is consistent with its ability to undergo posttranslational modifications (Prives and Hall, J. Pathol. 1999, 187:112-26). As the time following UV treatment increased, the distribution of the two forms changed and the slower migrating form became more abundant. These findings indicate that HEp-2 cells possess full length genomic copies of the p53 gene and suggest that the distribution of p53 changes in response to UV irradiation. However, even after stimulation by UV, the amount of p53 in the cells did not dramatically increase, implying that these cells likely contain a function that may facilitate the degradation of p53. Based on the phenotypic similarities between HEp-2 and HeLa cells, these results suggest that the p53 levels in the two cell lines may also be equivalent.

The next goal was to directly compare the amounts of the p53 present in the human tumor cells that were susceptible to oncoapoptosis. Equal amounts of HEp-2, 143B, and HeLa cell extracts were probed for their immune reactivities with an anti-p53 antibody that recognizes the human, monkey, and mouse forms of the protein. As a control, transformed African green monkey kidney Vero fibroblasts were also analyzed since these cells are resistant to apoptosis triggered by HSV-1 (Aubert and Blaho, supra). In addition, human embryonic kidney 293 cells that were transformed by exposing primary cells to fragments of sheared adenovirus type 5 DNA (Graham et al., J. Gen. Virol. 1977, 36:59-74) and primary mouse embryo fibroblasts (MEF) also were included. The 293 cells synthesized the highest level of p53 and the protein was equally distributed between a fast and a slow migrating form. This observation is consistent with the fact that the E1a protein present in the 293 cells functions to activate p53 (Lowe and Ruley, Genes Dev. 1993, 7:535-45). The slower migrating, modified form of p53 predominated in the Vero and MEF fibroblast cells. In contrast, the fast migrating form of p53 predominated in the HEp-2, 143, and HeLa cells. While the amounts of p53 detected in the HEp-2 and HeLa cells were substantially reduced compared to that in the 293 cells, the amount in the 143 cells was equivalent to the fast migrating form in the 293 cells. These results indicate that the cells which are sensitive to apoptotic death by rdHSV-1 strains synthesize reduced levels of p53 and this protein is markedly undermodified. These findings suggest that cells whose p53 has been inactivated are susceptible to killing via a viral oncoapoptotic mechanism.

### Example 2

### Primary Mouse and Human Fibroblasts are Resistant to Viral Oncoapoptosis but not Death Factor- or Death Receptor-Mediated Apoptosis 3

Experimental procedures and materials are described in Example 1. The results from immunoblots indicate that monkey and mouse fibroblasts synthesize predominantly phosphorylated p53, as evidenced by the presence of a slower migrating band. The Vero fibroblasts are resistant to the induction of apoptosis by HSV-1 (Aubert and Blaho, supra). To determine whether MEF cells are also insensitive, these cells were infected with either wt HSV-1 (KOS1.1) or rdHSV-1 (HSVΔ27 and HSVΔICP4. The amount of p53 synthesized in primary MEF cells was unaffected by infection with each virus strain. Wild type HSV-1 synthesized all classes of viral proteins, namely IE proteins ICP4 and ICP27, E protein TK, and L proteins gD and gC, indicating that MEF cells are permissive for viral replication. By contrast, the rdHSV-1 produced reduced amounts of the late gD protein and undetectable levels of gC. This pattern of viral protein accumulation is essentially identical to that observed following rdHSV-1 infection of Vero cells (Aubert and Blaho, J Virol. supra). These results are in contrast to that observed with rdHSV-1-infected HEp-2 cells where the synthesis of all classes of viral proteins are significantly reduced (Aubert and Blaho, supra).

To assess whether rdHSV-1 triggers apoptosis in infected MEF, cell morphologies were documented by phase-contrast microscopy and condensed nuclei were visualized following staining with a fluorescent Hoechst dye. Cells were also treated with staurosporine or TNF plus cycloheximide (CHX) to determine if these environmental agents could induce apoptosis in primary mouse fibroblasts. The concentrations of these environmental agents used in this study are sufficient to stimulate cell death in the human HEp-2 cells (Aubert et al., supra). Cells were analyzed 15 h post-infection. Consistent with the results from immunoblots described above, wild type HSV-1 (KOS1.1) infection led to the appearance of large, rounded cells showing classic (Aubert and Blaho, supra), cytopathic effect. In contrast, MEF cells infected with the rdHSV-l had a morphology identical to mock infected cells. Few, if any, cells with condensed nuclei were detected following infection with either rdHSV-1 strain. Based on these results we conclude that rdHSV-1 is unable to trigger apoptosis in primary mouse fibroblasts. Treatment of the MEF cells with either staurosporine or TNF plus CHX lead to a significant number of small apoptotic cells which contained condensed chromatin. These findings indicate that primary MEF cells are susceptible to the induction of cell death via both the death receptor and the death factor pathways. Taken together, the results show that the lack of cytopathic effect in MEF cells after rdHSV-1 infection is due to the inability of the virus to complete its replication cycle. Since the primary MEF cells contain predominantly phosphorylated p53, the conclusion is that the functional status of p53 plays a role in determining whether cells are susceptible to killing by an oncoapoptotic virus.

The transformed cell lines we analyzed above were of human origin. Thus, it was necessary to confirm our MEF cell results using a primary human cell line. We assessed the p53 status, the susceptibility of rdHSV-1 infection, and the ability to undergo apoptosis of primary human foreskin fibroblast (HFF) cells by immunoblotting, Hoechst staining, and DNA laddering assays. The p53 protein detected in the rdHSV-1- and wt (KOS1.1)-infected HFF cells was identical to that observed in mock infected cells. Consistent with observations using MEF cells, we found reduced levels of the late gD protein with no gC production during rdHSV-1 infection compared to wt HSV. This result demonstrates the inability of the rdHSV-1 strains to complete their replication cycles in HFF cells. However, we detected similar amounts of caspase-3 following mock, rdHSV-1 (HSVΔ27 and HSVΔ3), and wt HSV infection indicating that little to no death factor processing had occurred. We next performed ethidium bromide staining of low molecular weight DNA fragment preparations and did not detect significant laddering after wt or rdHSV-1 infection. We detected DNA fragmentation that migrated as a smear after treatment with staurosporine. Interestingly, the treatment with TNF plus CHX resembled the infected cells suggesting that HFF cells may be somewhat resistant to this environmental stimulator. Finally, we stained the infected and treated HFF cells with Hoechst DNA dye and visualized cellular and nuclear morphologies by fluorescence and phase-contrast microscopy. We detected little to no condensed chromatin following rdHSV-1 infection. The TNF plus CHX treated cells resembled the infected cells. Only the staurosporine treated cells showed features indicative of apoptosis. Based on these findings, we conclude that rdHSV strains cannot trigger apoptosis in primary human fibroblast cells.

### Example 3

### Adenoviral DNA-transformed Human Kidney 293 Cells are Resistant to Apoptosis

The results above indicate that cells whose p53 is undermodified are susceptible to apoptosis triggered by rdHSV-1 while those which accumulate phosphorylated p53 are resistant. To confirm the prediction that the amount of phosphorylated p53 present in a given cell is an accurate indicator of whether the cell will be killed by an rdHSV-1, 293 cells were analyzed. Human 293 cells were infected with wt and rd HSV-1 strains in the presence or absence of the translation inhibitor CHX. This inhibitor was added as a control since HEp-2 cells infected with wt or rdHSV-1 in the presence of CHX are apoptotic (Aubert and Blaho, supra; Aubert et al., supra). Staurosporine was also added in this study as an additional positive control for the induction of apoptosis (Aubert et al., supra). Following the infections and treatments, immunoblots were performed for the PARP and caspase-3 death factors, as well as for p53 and p105^{RB} proteins. Unprocessed PARP and caspase-3 was detected in all case indicating that the cells did not undergo apoptosis. Consistent with the results above, approximately equal amounts of the two forms (faster migrating and slower migrating) of p53 were detected under all conditions. Slightly reduced total amounts of p53 were seen in the wt and rdHSV (cGalΔ3, but not vBSΔ27) infections in the absence of CHX. In addition, two forms of p105^{RB} were observed after all treatments. These results indicate that these infection and treatment conditions are insufficient to trigger apoptosis in 293 cells.

To confirm that 293 cells are resistant to apoptosis, the cells were either UV irradiated, serum starved, or treated with TNF plus CHX or a double dose of STS prior to performing immunoblots for p53 and death factors. Even under this diverse array of stimulation, no signs of apoptosis were detected, as evidenced by the presence of a band representing unprocessed PARP. Approximately equal amounts of the two forms of p53 were detected under all conditions. There appeared to be a slight increase in the total amount of p53 following UV treatment, suggesting that this irradiation may have induced the synthesis of p53. These results indicate that 293 cells are unable to be induced to undergo apoptosis. As indicated earlier, human 293 cells are derived from primary embryonal kidney cells and were transformed by DNA from adenovirus type 5 (Graham et al., supra). While the E1a protein in 293 cells activates and stabilizes p53, apoptosis is prevented through the action of the E1b proteins (Debbas and White, Genes Dev. 1993, 7:546-54; Lowe and Ruley, Genes Dev. 1993, 7:535-45). The E1b 19kDa protein functionally substitutes for the activity of Bcl-2 while the E1b 55 kDa binds p53 and inhibits its function. Based on this results, the inventors conclude that cells which contain viral anti-apoptotic activities, such as those conferred by the adenovirus E1b region, are resistant to rdHSV-1 induced cell death.

Finally, immunoblots for various viral proteins were performed following wt and rdHSV-1 infection of the 293 cells. As observed with MEF cells, all viral proteins (ICP4, ICP27, TK, gD and gC) were observed with wt HSV-1 while reduced gD and no gC was made by the rdHSV-1. Identical results were obtained using the related 293T cells. Taken together, these results show that human 293 are resistant to the induction of apoptosis by rdHSV-1. However, in contrast to primary MEF cells, 293 cells are also resistant to the induction of apoptosis by a wide variety of environmental stimulators. These findings support the finding that cells which produce a significant amount of slow migrating p53 are unable to be killed by HSV-1 induced apoptosis.

Thus, the inventors have shown that three human tumor cell lines are susceptible to death by rdHSV-1 induced apoptosis. The common feature among the susceptible cells was that they accumulated undermodified p53. This finding suggests that the modified state of p53 may be a marker for susceptibility to apoptosis triggered by rdHSV-1. In addition, the levels of p53 in the sensitive cells were much less than that in an oncogene transformed cell.

The present invention shows that herpes simplex viruses that are unable to complete their replication cycle initiate an abortive infection that triggers tumor cells to undergo apoptosis. These results have significant implications in the development of anticancer therapies and may be used in the treatment of drug resistant cancers.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed:

1. A method of inducing apoptosis of a cancer cell, which method comprises contacting the cancer cell with a composition comprising an herpes simplex virus (HSV) having a defect in ICP27 (HSVΔ27), wherein the cancer cell is resistant to anti-cancer therapy.

2. A method of sensitizing a tumor to anti-cancer therapy, which method comprises contacting the tumor with a composition comprising HSVΔ27, wherein the tumor is more sensitive to treatment with an anti-cancer agent when contacted with the composition comprising HSVΔ27 than in the absence of the contacting with the HSVΔ27.

3. The method of claim 2, wherein the anti-cancer therapy is selected from the group consisting of a chemotherapeutic agent and radioactive agent.

4. The method of claim 1, wherein the cancer cell is part of a solid tumor.

5. The method of claim 2, wherein the tumor is contacted with the HSVΔ27 before the anti-cancer therapy.

6. A method of inducing apoptosis of a solid tumor cancer cell, wherein the cancer cell is derived from a tissue, organ, or cell selected from the group consisting of brain, lung, liver, spleen, kidney, lymph node, small intestine, blood cells, pancreas, colon, stomach, breast, endometrium, prostate, testicle, ovary, cervix, skin, head and neck, esophagus, laryx, bone, bone marrow and blood, which method comprises contacting the cancer cell with a composition comprising a herpes simplex virus (HSV) having a defect in ICP27 (HSV∇27), and wherein the cancer cell is resistant to anti-cancer therapy.

7. A method of sensitizing a tumor to an anti-cancer therapy selected from the group consisting of a chemotherapeutic agent, a radioactive agent, a nucleic acid encoding a cancer therapeutic agent or an anti-angiogenic agent, which method comprises contacting the tumor with a composition comprising HSV∇27, wherein the tumor is more sensitive to treatment with an anti-cancer agent when contacted with the composition comprising HSV∇27 than in the absence of the contacting with the HSV∇27.

8. A method of inducing apoptosis of a cancer cell, which method comprises contacting the cancer cell with a HSVΔ27, wherein the cancer cell comprises a defect in p53.

9. The method of claim 8, wherein the defect in p53 comprises a mutation in the p53 gene.

10. The method of claim 8, wherein the defect in p53 is a defect in the expression of p53 of the cells.

11. The method of claim 8, wherein the defect in p53 comprises a decrease in the post-translational phosphorylation of p53 yielding an under-phosphorylated p53 as compared to wild-type p53 in non-cancer cells.

12. The method of claim 8 wherein the defect in p53 comprises an increase in the post-translational phosphorylation of p53 yielding an over-phosphorylated p53 as compared to wild-type p53 in non-cancer cells.

13. A method of inducing apoptosis of a cancer cell comprising contacting the cancer cell with a HSV∇27, wherein the cancer cell comprises a defect in p53 selected from the group consisting of a mutation in the p53 gene, a defect in the expression of p53 of the cells, a decrease in the post-translational phosphorylation of p53 yielding an under-phosphorylated p53 as compared to wild-type p53 in non-cancer cells, and an increase in the post-translation phosphorylation of p53 yielding an over-phosphorylated p53 as compared to wild-type p53 in non-cancer cells.

14. The method of claim 1, 2, or 8 wherein the HSVΔ27 further comprises a defect in an additional immediate early (IE) gene, which HSVΔ27 triggers apoptosis but does not prevent apoptosis.

15. The method of claim 14, wherein the additional IE gene is ICP4 and gD.

16. The method of claim 14, wherein the HSVΔ27 further comprises a defect in ICP4.

17. The method of claim 14, wherein the cancer cell is derived from a tissue, organ, or cell selected from the group consisting of esophagus, larynx, cervix, bone and kidney.

18. A method of treating cancer in a mammal, which method comprises administering to the mammal a composition comprising HSVΔ27 in an amount effective to induce apoptosis of cancer cells in the mammal.

19. The method of claim 18, wherein the mammal is a human.

20. The method of claim 18, wherein the mammal has a cancer of a tissue, organ, or cell selected from the group consisting of esophagus, larynx, cervix, bone, and kidney.

21. The method of claim 18, wherein the cancer is in the form of an operable tumor, and the administering is performed after the tumor is resected.

22. The method of claim 21, wherein the tumor is resistant to cancer therapy.

23. The method of claim 21, wherein the tumor comprises cells that have a defective p53.

24. The method of claim 21, wherein the HSVΔ27 is administered to the mammal locally at the site of the tumor.

25. The method of claim 21, wherein the HSVΔ27 is administered to the mammal systemically.

26. The method of claim 18, further comprising administering an anti-cancer therapy, wherein the anti-cancer therapy is selected from the group consisting of a chemotherapeutic agent and radiation.

27. The method of claim 26, wherein the anti-cancer agent is administered after administration of the HSVΔ27.

28. The method of claim 26, wherein the anti-cancer agent is administered to the mammal locally at the site of the cancer.

29. The method of claim 26, wherein the chemotherapeutic agent is selected from the group consisting of cisplatin, 5-FU, mitomycin, etoposide, camptothecin, actinomycin-D, doxorubicin, verapamil, podophyllotoxin, daunorubicin, vincristine, vinblastine, melphalan cyclophosphamide, TNF-α, taxol, and bleomycin.

30. The method of claim 18, wherein the HSVΔ27, is injected directly into the tumor.

31. The method of claim 26, wherein the radiation therapy comprises administering X-irradiation, UV-radiation, γ-radiation, or microwave radiation.

32. The method of claim 31, wherein the total dose of radiation is about 1 Gy to about 80 Gy.

33. The method of claim 31, wherein the total does of x-ray radiation is between 200 and 6000 roentgens.

34. A method of inducing apoptosis of a cancer cell, which method comprises contacting the cancer cell with a herpes simplex virus (HSV) having a defect in ICP4 (HSVΔICP4), wherein the cancer cell is resistant to anti-cancer therapy.

35. A method of sensitizing a tumor to anti-cancer therapy, which method comprises contacting the tumor with a composition comprising HSVΔICP4 wherein the tumor is more sensitive to treatment with an anti-cancer agent when contacted with the composition comprising HSVΔICP4 than in the absence of the contacting with the HSVΔICP4.

36. A method of inducing apoptosis of a cancer cell, which method comprises contacting the cancer cell with a HSVΔICP4, wherein the cancer cell comprises a defect in p53.

37. A method of inducing apoptosis of a solid tumor cancer cell, wherein the cancer cell is derived from a tissue, organ, or cell selected from the group consisting of brain, lung, liver, spleen, kidney, lymph node, small intestine, blood cells, pancreas, colon, stomach, breast, endometrium, prostate, testicle, ovary, cervix, skin, head and neck, esophagus, larynx, bone, bone marrow, and blood, which method comprises contacting the cancer cell with a composition comprising a herpes simplex virus (HSV) having a defect in ICP4 (HSVΔICP4), and wherein the cancer cell is resistant to anti-cancer therapy.

38. A method of sensitizing a tumor to an anti-cancer therapy selected from the group consisting of a chemotherapeutic agent, a radioactive agent, a nucleic acid encoding a cancer therapeutic agent or an anti-angiogenic agent, which method comprises contacting the tumor with a composition comprising HSVΔICP4, wherein the tumor is more sensitive to treatment with an anti-cancer agent when contacted with the composition comprising HSVΔICP4 than in the absence of the contacting with the HSVΔICP4.

39. A method of treating cancer in a mammal, which method comprises administering to the mammal a composition comprising HSVΔICP4 in an amount effective to induce apoptosis of cancer cell in the mammal.

40. A method of inducing apoptosis of a cancer cell comprising contacting the cancer cell with a HSVΔICP4, wherein the cancer cell comprises a defect in p53 selected from the group consisting of a mutation in the p53 gene, a defect in the expression of p53 of the cells, a decrease in the post-translational phosphorylation of p53 yielding an under-phosphorylated p53 as compared to wild-type p53 in non-cancer cells, and an increase in the post-translational phosphorylation of p53 yielding an overphosphorylated p53 as compared to wild-type p53 in non-cancer cells.
